(19) United States
(12) Patent Application Publication
(10) Pub. No.: US 2025/0254405 A1
(43) Pub. Date: Aug. 7, 2025

(54) ENDOSCOPE HAVING SIMULTANEOUS MULTI-MODAL IMAGING

(71) Applicant: Verily Life Sciences LLC, Dallas, TX (US)

(72) Inventors: Kirk Gossage, Montara, CA (US); Tamara Troy, San Francisco, CA (US)

(21) Appl. No.: 18/855,158

(22) PCT Filed: Apr. 27, 2023
(86) PCT No.: PCT/US2023/020274

§ 371 Date: Oct. 8, 2024

Related U.S. Application Data
(60) Provisional application No. 63/341,141, filed on May 12, 2022.

(51) Int. Cl. H04N 23/16; A61B 1/04; A61B 1/06; H04N 23/11

(52) U.S. Cl. CPC H04N 23/16; A61B 1/043; A61B 1/0638; A61B 1/0646; A61B 1/0655; H04N 23/11; A61B 2562/04

(57) ABSTRACT

An imaging system includes an endoscope tube, an illumination system, first and second image sensors, and a controller. The illumination system is coupled to the endoscope tube and configured to emit first illumination light having a first wavelength profile and excitation light having an excitation wavelength profile outside of the first wavelength profile. The first image sensor is aligned with a first filter configured to pass first image light, received in response to the first illumination light, to the first image sensor and to block the excitation light. The second image sensor is aligned with a second filter configured to pass fluorescence light, emitted in response to the excitation light, to the second image sensor. The controller includes logic to simultaneously illuminate a scene with the first illumination light and the excitation light and capture first image data and fluorescence image data with the first and second image sensors.

### CROSS-REFERENCE TO RELATED APPLICATIONS

This application claims the benefit of U.S. Provisional Application 63/341,141, filed May 12, 2022, which is hereby incorporated by reference in its entirety.

### TECHNICAL FIELD

This disclosure relates generally to imaging systems, and, in particular, but not exclusively, relates to endoscopes.

### BACKGROUND INFORMATION

It can be advantageous to illuminate a scene with two or more different lights having different wavelengths to acquire more information about the scene. For example, fluorescence imaging is an imaging technique that utilizes fluorophores for a variety of applications, such as visualizing biological processes, cancer detection, marking or tracking biological features, and the like. Visible light spectroscopy (VLS) is also used to visualize biological processes, such as mucosal oxygen saturation during gastrointestinal endoscopy, and can also be used to show visible color information of a scene being imaged.

Conventional imaging systems require a user to acquire separate, temporally sequenced, images distinctly illuminated at different wavelength profiles. Because both images are captured with a common image sensor having common configuration settings, the overall image quality suffers. The image sensor can be adjusted to provide a more optimal image of the light at a first wavelength, but does so, necessarily, at the detriment of the image at the second wavelength. For example, in order to capture a visible image in an optimal manner, a different frame rate or exposure setting may be needed than to capture a fluorescence image. Conventional imaging systems also require that only a single illumination band may illuminate a scene at a time. Since the image sensor configuration settings cannot be changed when quickly interleaving between each imaging modality, one or both of the imaging modalities are imaged with compromised settings.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described.

FIG. 1A is an illustration of an endoscope system capable of simultaneous multi-modal imaging, in accordance with an embodiment of the disclosure.

FIG. 1B is an illustration of an endoscope capable of simultaneous multi-modal imaging, in accordance with an embodiment of the disclosure.

FIG. 2A is an illustration of an endoscope tube capable of simultaneous multi-modal imaging, where the image sensors are located on the distal end of the endoscope tube, in accordance with an embodiment of the disclosure.

FIG. 2B is an illustration of an endoscope tube capable of simultaneous multi-modal imaging, including a first and second reflector for directing light onto the image sensors, in accordance with an embodiment of the disclosure.

FIG. 2C is an illustration of an endoscope tube capable of simultaneous multi-modal imaging, where the image sensors are located at the proximal end of the endoscope tube and including a beam splitter to direct light onto the image sensors, in accordance with an embodiment of the disclosure.

FIG. 3 is a flowchart of a process of simultaneous multi-modal imaging, in accordance with an embodiment of the disclosure.

FIG. 4 illustrates an example scenario of simultaneous capture of multi-modal light from a scene, in accordance with an embodiment of the disclosure.

### DETAILED DESCRIPTION

Embodiments of a system and method for simultaneous multi-modal imaging with an endoscope are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In general, embodiments of the present disclosure are described in the context of imaging using an endoscope in a surgical setting. However, it should be appreciated that techniques and embodiments described herein are generally applicable to the field of imaging and image processing techniques and thus should not be deemed limited to only endoscopic imaging and/or surgical settings. For example, techniques described herein may be used for image processing of any illuminated images. In the same or other embodiments, imaging may be utilized outside of a surgical setting. Additionally, one of ordinary skill in the art will appreciate imaging covers a variety of areas including, but not limited to microscopy, imaging probes, spectroscopy, and the like. That said, the simultaneous multi-modal imaging schemes described herein are particularly well-suited for fluorescence imaging using a fluorescent dye, such as indocyanine green (ICG), for medical diagnostic imaging.

It can be advantageous to illuminate a scene with two or more lights having different wavelengths, or set of wavelengths, and/or intensities. Conventional technology requires a user to illuminate a scene with a single light source at a time. A user must illuminate the scene with a first light having a first wavelength band, turn off the first light, and then illuminate the scene with a second light having a second wavelength band outside of the first wavelength band. The acquired first image and second image are then interleaved to give the impression that they are taken contemporaneously. Further, conventional systems only include a single set of image sensors to capture all light received from the scene at the same wavelength with the same settings. In order to achieve an optimal image, an image acquisition characteristic, such as frame rate, duty cycle, or exposure may need to be adjusted. However, because a single image sensor is used to capture both the first and second image data, adjusting the image sensor to produce an optimal first image is at the expense of the quality of the second image.

The device and techniques disclosed herein provide a solution that allows a user to simultaneously illuminate a scene with two or more illumination sources having different wavelengths, intensities, or other illumination characteristics. The illumination sources may have different wavelength profiles. As described herein, a wavelength profile may be a wavelength band or a set of wavelengths, and may be discrete or continuous. Additionally, a wavelength profile may be monochromatic, or prismatic (i.e. multi-colored). Two filters may be aligned with two image sensors, the two filters configured to pass a desired light to the image sensor and block other unwanted light. Because of this, each image sensor can be independently adjusted in real-time to achieve simultaneous multi-modal imaging. A frame rate, refresh rate, gain, and/or exposure can be adjusted for each image sensor independently of the other, to achieve an optimal image with a first light, and, for example, a fluorescent light, simultaneously. The gain may be either analog or digital gain. Further, two or more illumination systems can be adjusted to emit two or more lights having different wavelength profiles contemporaneously, and a user can adjust the intensity, duty cycle, or wavelength profile of said two or more lights independently and contemporaneously to the images being acquired and displayed on a screen.

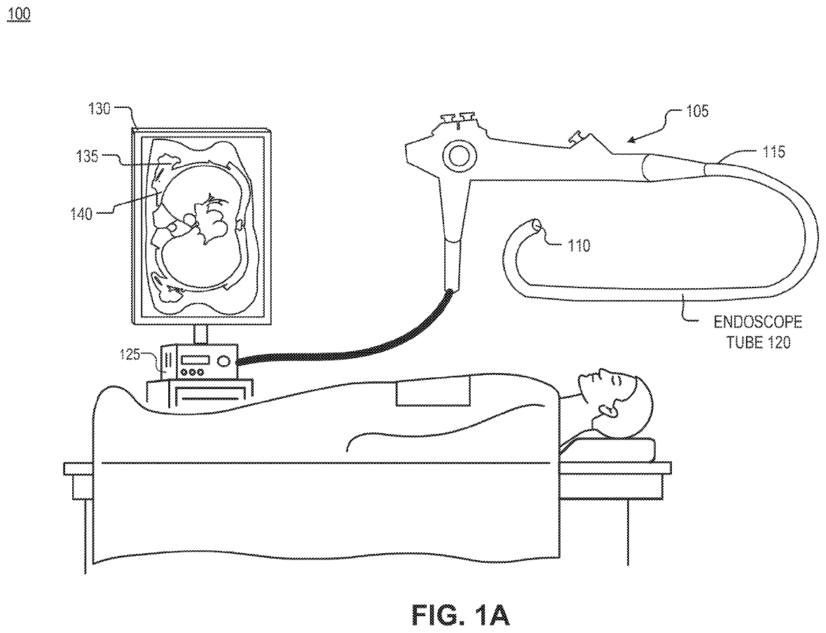

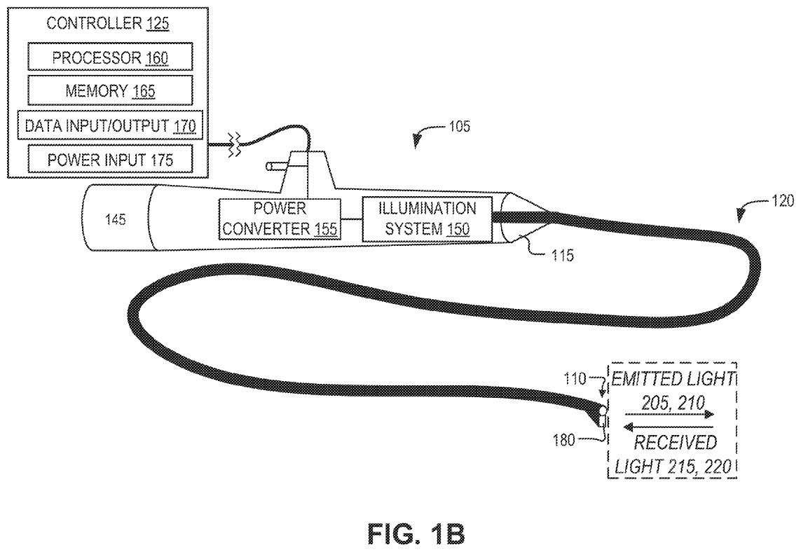

FIG. 1A is an illustration of an endoscope system 100 capable of simultaneous multi-modal imaging, in accordance with an embodiment of the disclosure. System 100 includes an endoscope 105, a controller 125, and a display 130. The endoscope 105 may include an endoscope tube 120, having a distal end 110 and a proximal end 115. The endoscope 105 may be communicatively coupled to controller 125 to output, on the display 130, images or videos of a scene (e.g., an internal or external view of the patient) in real-time. For example, system 100 may capture both a fluorescence image 135 and a color image 140 at the same time, to enable a user to view, in real-time, a combined image showing both a fluorescence image 135, which typically is visualized outside of the visible wavelength range of light, and a color image 140. The controller 125 may implement various image processing techniques described herein to enable simultaneous multi-modal imaging.

As illustrated, the proximal end 115 of the endoscope 105 may have a number of buttons or joysticks to control movement of the distal end 110. One of ordinary skill in the art will appreciate that endoscope 105 depicted here is merely a cartoon illustration of an endoscope, and that the term “endoscopy” should encompass all types of endoscopy (e.g. laparoscopy, bronchoscopy, cystoscopy, colonoscopy, sigmoidoscopy, thoracoscopy, laryngoscopy, arthroscopy, robotic surgery, or any other situation when a camera or optical probe is used), and that an endoscope may include at least “chip-on-tip” devices, rod lens devices, image fiber devices, and the like. It is further appreciated that endoscope 105 may also be included in or otherwise coupled to a surgical robotic system.

FIG. 1B is an illustration of an endoscope 105 capable of simultaneous multi-modal imaging, in accordance with an embodiment of the disclosure. The endoscope 105 includes an endoscope tube 120 with a distal end 110 and a proximal end 115, and a housing 145. The endoscope 105 may include one or more illumination systems 150, and a power converter 155. The endoscope 105 is coupled to a controller 125, which includes a processor 160, memory 165, data input/output 170, and power input 175. The endoscope may further include one or more image sensors 180 positioned proximate to the distal end 110 of the endoscope tube 120.

Endoscope 105 includes a proximal end 115 that may be hand-held, or mounted, and a distal end 110 configured to be inserted into a patient receiving a surgical procedure. In some embodiments, the illumination system 150 includes one or more light emitting diodes (LEDs), one or more laser diodes, or the like. Illumination system 150 is optically coupled to the proximal end 115 of the endoscope tube 120 to emit light 205, 210. In some embodiments, emitted light 205, 210 includes a first illumination light 205 having a first wavelength profile and an excitation light 210 having a second wavelength profile, distinct from the first wavelength profile, as described herein. In some embodiments, the first illumination light 205 is visible light, having a wavelength profile within the visible spectrum while the excitation light 210 can span from ultraviolet to infrared light intended to excite fluorescence or some other form of stimulated emission. In some embodiments, excitation light 210 may even include red wavelengths.

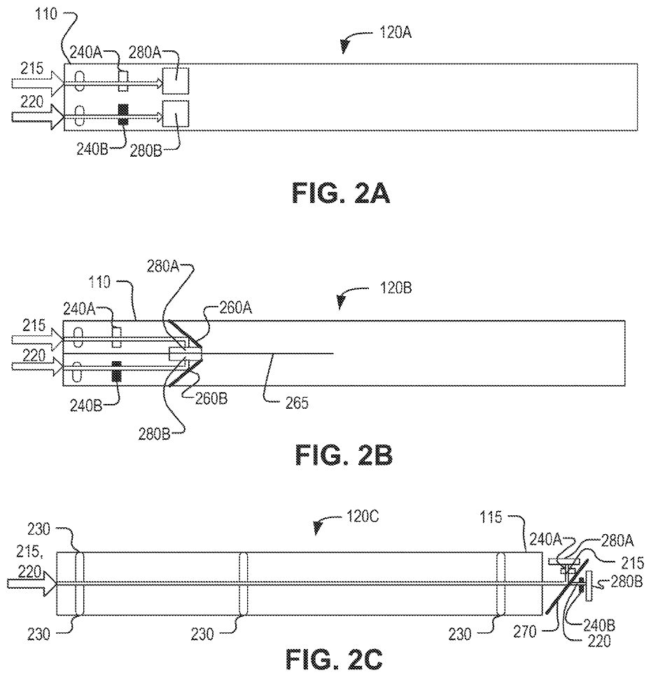

One or more optical fibers, as shown in FIGS. 2A-2C, may be bundled within the endoscope tube 120 to optically couple the illumination system 150 with the distal end 110 of the endoscope tube 120 to allow for the distal end 110 to be positioned to a scene being imaged (e.g., surgical site within a body cavity). Image sensor 180 may be coupled to the distal end 110 of the endoscope tube 120 and positioned to receive received light 215, 220 to capture simultaneous multi-modal images and/or videos representative of the scene. Received light 215, 220 may include a first image light 215 and a fluorescence light 220, as described herein.

It is appreciated that in some embodiments, the image sensor 180 is not disposed proximate to the distal end 110 of the endoscope tube 120. Rather in some embodiments, the image sensor 180 is disposed within the housing 145 of the endoscope 105 or the proximal end 115 of the endoscope 105. In one embodiment, endoscope 105 includes one or more waveguides (e.g., optical fibers) disposed within the endoscope tube 120, with a first portion of the optical fibers coupled to the illumination system 150 to direct emitted light 205, 210 from the illumination system 150 through the endoscope tube 120 and out the distal end 110 and a second portion of the optical fibers coupled to the image sensor 180 to direct received light 215, 220 received at the distal end 110 through the endoscope tube 120 and to the image sensor 180.

Controller 125 may be disposed within the housing 145 of the endoscope tube 105, or external (e.g., wired or wirelessly connected) to endoscope 105. Controller 125 includes a processor 160, memory 165 (e.g., any non-transitory computer-readable storage medium or machine accessible storage medium), data input/output 170 (e.g., to send/receive the images and/or video from image sensor 180), and power input 175 (e.g., to power endoscope 105). Data input/output 170 may include an input apparatus coupled to controller 125. The input apparatus may be positioned to receive an input command from an operator, such as a surgeon. In response to receiving the input command, the endoscope may perform simultaneous imaging in multiple modalities, including fluorescence imaging and visible light imaging. The controller 125 may be coupled to the illumination system 150, the image sensor 180, and memory 165. The memory 165 includes instructions that when executed by the controller 125 cause the system (such as system 100 of FIG. 1A) to perform operations for simultaneous multi-modal imaging.

It is appreciated that the controller 125 may orchestrate operation of the imaging system capable of simultaneous multi-modal imaging 100 of FIG. 1A and includes software (e.g. instructions included in memory 165 coupled to processor 160) and/or hardware logic (e.g. application specific integrated circuits, field-programmable gate arrays, and the like) that when executed by the controller 125 causes the system 100 of FIG. 1A to perform operations for simultaneous multi-modal imaging, in accordance with embodiments of the disclosure.

FIG. 2A is an illustration of an endoscope tube 120A capable of simultaneous multi-modal imaging, where image sensors 280A, 280B are located on the distal end 110 of the endoscope tube 120A, in accordance with an embodiment of the disclosure. The first filter 240A is aligned with the first image sensor 280A, and the second filter 240B is aligned with the second image sensor 280A.

Because first image light 215 and fluorescence light 220 are returned to the endoscope tube 120 simultaneously, it should be understood that while FIGS. 2A and 2B illustrate the first image light 215 and the fluorescence light 220 separately, for clarity, the first image light 215 and the fluorescence light 220 are collectively referred to as scene light.

A first filter 240A may be aligned with the first image sensor 280A. The first filter 240A is configured to pass first image light 215, received in response to the first illumination light, to the first image sensor 280A and to block the fluorescence light 220, and the excitation light. A second filter 240B is configured to pass the fluorescence light 220 while substantially blocking both the first image light 215 and the excitation light and first illumination light. In some embodiments, the second filter is a near-infrared (NIR) long-pass filter having a cutoff wavelength longer than the excitation wavelength (e.g., 780-800 nm), meaning that it is configured to remove all wavelengths, such as UV and visible light, shorter than a desired NIR cutoff wavelength. In some embodiments, the long-pass filter has a cutoff point at 800 nm. In this way, the second filter 240B may pass the fluorescence light 220 longer than 800 nm while blocking the visible image light 215 and excitation light below 800 nm.

FIG. 2B is an illustration of an endoscope tube 120B capable of simultaneous multi-modal imaging, including a first and second reflector 260A, 260B for directing received light 215, 220 onto the image sensors 280A. 280B, in accordance with an embodiment of the disclosure. In the illustrated embodiment, the first and second image sensors 280A, 280B are located at the distal end 110, but include light sensitive arrays pointing perpendicular to a longitudinal axis 265 of an endoscope tube 120. In such embodiments, the first and second reflectors 260A, 260B are positioned to bend received light 215, 220 received along the longitudinal axis 265 onto the light sensitive arrays of the image sensors 280A, 280B. In some embodiments, the received light 215, 220 pass through the first and second filter 240A, 240B before being bent by the first and second reflectors 260A, 260B. Accordingly, the first filter 240A may filter the received light 215 before it reaches the first image sensor 280A. Likewise, the second filter 240B may filter the received light 220 before it reaches the second image sensor 280B.

FIG. 2C is an illustration of an endoscope tube 120C capable of simultaneous multi-modal imaging, where the image sensors 280A, 280B are located on the proximal end 115 of the endoscope tube 120, in accordance with an embodiment of the disclosure. In this embodiment, one or more optical fibers 230 are bundled within the endoscope tube 120 to optically couple received scene light onto the image sensors 280A, 280B located on the proximal end 115 of the endoscope tube 120C. In the illustrated embodiment, first image sensor 280A is oriented horizontally, so that it is parallel with the endoscope tube 120 and the second image sensor 280B is oriented vertically, so that it is perpendicular to the first image sensor 280A. A beam splitter 270 may be located at the proximal end 115 of the endoscope tube 120, between the horizontally oriented first image sensor 280A and the vertically oriented second image sensor 280B so that light 215, 220 is split and directed onto both image sensors 280A, 280B. The beam splitter 270 may be implemented as a dichroic multi-layer film. In other embodiments, the beam splitter 270 is a polarizing beam splitter. In some embodiments, the beam splitter 270 is configured to receive the first image light 215 and the fluorescence light 220 and direct the first image light 215 to the first image sensor 280A, and the fluorescence light 220 to the second image sensor 280B.

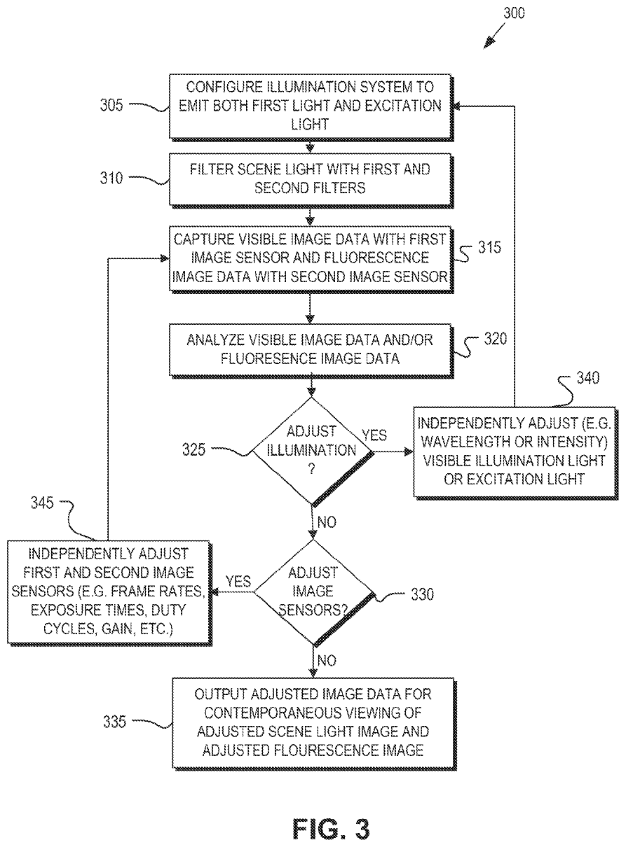

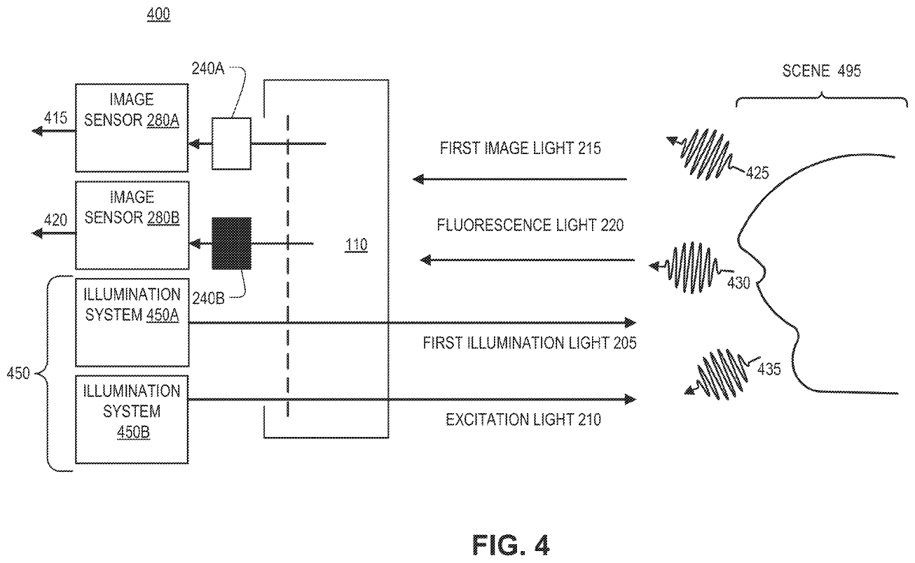

FIG. 3 is a flowchart of a process 300 of simultaneous multi-modal imaging, in accordance with an embodiment of the disclosure. Process 300 may be implemented by fluorescence imaging system 100 illustrated in FIG. 1A to perform simultaneous multi-modal imaging. Process 300 is described with reference to FIG. 4. It is appreciated that the order in which some of the process blocks appear in process 300 should not be deemed limiting. Rather one of ordinary skill in the art having the benefit of the present disclosure will understand that some of the process blocks may be executed in a variety of orders not illustrated, or even in parallel.

Process 300 begins in process block 305, where the illumination system 450 is configured to emit both a first illumination light 205 and an excitation light 210. In the illustrated embodiments the illumination system 450 includes two illumination systems 450A, 450B: one for emitting first illumination light 205, and one for emitting excitation light 210, as shown in FIG. 4. In some embodiments, the first illumination light 205 may be visible light. In some embodiments, the first illumination light 205 is white light, but the first illumination light may be any light having a wavelength profile within the visible spectrum. In some embodiments, the first illumination light 205 may be a broad spectrum light that spans the visible range, discrete spectra within the visible range (e.g., multiple lasers with different narrow band spectra), or other light that may be illuminated simultaneously or sequentially for generating a color image of the scene. In some embodiments, the excitation light 210 is a light having a wavelength profile sufficient to induce fluorescence from scene 495. As described herein, the illumination systems 450A, 450B are capable of emitting the first illumination light 205 and the excitation light 210 simultaneously. In some embodiments, the illumination systems 450A, 450B may be configured by providing power to activate an appropriate laser, LED, or other light emission source.

In block 310, scene light is filtered with a first and second filter 240A, 240B. The scene light refers to the light received from scene 495 in response to the illumination and refers collectively to first image light 215 and fluorescence light 220. In some embodiments, the first filter 240A is configured to pass the first image light 215 to the first image sensor 280A, while blocking the fluorescence light 220. In some embodiments, the second filter 240B is configured to pass the fluorescence light 220 to the second image sensor 280B, while blocking the first image light 215 and the excitation light 210. In some embodiments, the second filter 240B is a long-pass filter, configured to having a cutoff wavelength longer than the excitation wavelength as described herein.

In block 315, visible image data 415 and fluorescence image data 420 is captured by the first image sensor 280A and the second image sensor 280B, respectively. In some embodiments, the visible image date 415 is representative of the first image light 215, and the fluorescence image data 420 is representative of the fluorescence light 220. The visible image data and the fluorescence image data may be spatially associated. For example, a pixel in column 1 row 1 of a fluorescence image and a visible image may both be representative of the same spatial portion of a scene 495.

In block 320, the visible image data 415 and/or the fluorescence image data 420 is analyzed. In some embodiments, the visible image data 415 and/or the fluorescence image data 420 is analyzed by controller 125. In some embodiments, the visible image data 415 and/or the fluorescence image data 420 is reviewed by an operator of the endoscope (e.g., a surgeon), after displaying the visible image 135 and the fluorescence image 140 contemporaneously on display 130 illustrated in FIG. 1A.

In decision block 325, if the operator of the endoscope, or the processor of the endoscope determines the illumination needs to be adjusted, the process 300 proceeds to block 340. In block 340, the first illumination light 205, the excitation light 210, or both, are independently adjusted. In some embodiments, adjusting the illumination includes adjusting the wavelength, intensity, duty cycle, or gain of the first illumination light 205 and/or the excitation light 215. These illumination characteristics of the first illumination light 205 and/or the excitation light 215 can be adjusted independently because of the two illumination systems 250A, 250B described herein. The operator, or the processor of the endoscope, can adjust the wavelength and/or intensity of the first illumination light 205, while holding the wavelength and/or intensity of the excitation light constant 215, and vice versa. The adjustment of the illumination can be done while emitting both the first illumination light 205 and the excitation light 215 contemporaneously. After the operator or the processor has adjusted the first illumination light 205 and/or the excitation light 215, the process 300 proceeds back to block 305.

Returning to decision block 325, if the operator of the endoscope or controller 125 determine that the illumination does not need to be adjusted, the process 300 proceeds to decision block 330. In decision block 330, if the operator or controller 125 determine that the image sensors 280A, 280B need to be adjusted, the process 300 proceeds to block 345. In block 345, the first and second image sensors 280A, 280B are adjusted. In some embodiments, adjusting the image sensors involves adjusting an image acquisition characteristic of a selected one of the first or second image sensors independently between the first and second image sensors. The image acquisition characteristics include, but are not limited to, a frame rate, an exposure time, or a duty cycle. In operation, each image acquisition characteristic can be adjusted independently between the first and second image sensors. For example, at least one of a first frame rate or a first exposure time of the first image sensor 280A can be adjusted while holding a second frame rate and a second exposure time of the second image sensor 280B constant during acquisition of a series of visible images and fluorescence images with the first and second image sensors, 280A, 280B respectively. Similarly, an operator can also adjust at least one of the second frame rate or the second exposure time of the second image sensor 280B while holding the first frame rate and first exposure time of the first image sensor 280A constant while acquiring a series of visible images and fluorescence images, with the first and second image sensors, respectively. For example, a visible color image may have optimal quality at a frame rate of 60-160 FPS, while an Indocyanine Green (ICG) image may require a frame rate at 12-60 FPS. Additionally, an optimal visible color image may only need 25-50% of the duty cycle of an optimal ICG image. When the operator or the processor of the endoscope determines that the first and second image sensors have been adjusted as needed, the process 300 proceeds to block 315.

Returning to decision block 330, if the operator or controller 125 choose not to adjust the image sensors, the process 300 proceeds to block 335. In block 335, the adjusted image data is output for contemporaneous viewing of the adjusted visible image and the adjusted fluorescence image. For example, the fluorescence image may be superimposed over the visible image. In some embodiments, the adjusted image data is output on a display, such as display 125 illustrated in FIG. 1A. In some embodiments, the visible image or the fluorescent image is not adjusted while the other image is. It should be appreciated that each block of process 300 proceeds effectively contemporaneously, meaning that the output of the adjusted image data is updated in real-time, as the illumination system, the image sensors, or both are adjusted.

FIG. 4 illustrates an example scenario 400 of simultaneous capture of multi-modal light from a scene 495, in accordance with an embodiment of the disclosure. The example scenario 400 includes a distal end 110 of an endoscope (e.g., endoscope 105 of FIGS. 1A-1B), including a first illumination system 450A, a second illumination system 450B, a first filter 240A, a second filter 240B, a first image sensor 280A, and a second image sensor 280B. It is appreciated that the illumination systems 450A, 450B and the image sensors 280A, 280B correspond to the like named elements illustrated in FIGS. 1A-2C.

The first illumination system 450A may be configured to provide illumination to the scene 495 by emitting a first illumination light 205 (e.g. providing power to one or more light emitting diodes, laser diodes, or the like with wavelengths within the visible spectrum of light). The first illumination light 205 subsequently reflects/scatters from the scene 495 as first image light 215 and captured by the first image sensor 280A as visible image data 415. In some embodiments, first illumination light 205 is visible light. In some embodiments, the first illumination light 205 includes a light with a wavelength between 380 nm and 750 nm. For example, the first illumination system 450A may include a plurality of laser diodes (e.g., a combination of a 450 nm laser, a 520 nm laser, a 550 nm laser, and a 650 nm laser) that may be powered or otherwise activated to simulate white light for color imaging of the scene 495. In other embodiments, the first illumination light 205 is also an excitation light having a wavelength profile outside the wavelength profile of excitation light 210. For example, the first illumination light 205 may be infrared (IR) light or ultraviolet (UV) light.

In the illustrated embodiment, first filter 240A is placed between the first image sensor 280A and scene 495 to prevent fluorescent light 220, excitation light 210, and other stray light 425 from reaching the first image sensor 280A. The first image sensor 280A then outputs first image data 415 to controller 125.

The fluorescence light 220 may be captured simultaneously with the first image light 215 (e.g., to capture a combined image showing a color image 135 and fluorescence image 140, as illustrated in FIG. 1A). The second illumination system 250B may be configured (e.g. by adjusting a power to one or more lasers included in the second illumination system 250B) to provide illumination of the scene 495 by emitting an excitation light 210. The excitation light 210 includes an excitation wavelength profile. In some embodiments, the excitation wavelength profile is between 780-800 nm, but it may also be any wavelength that induces fluorescence based on the chemical composition of the fluorophores included into the scene 495. The excitation wavelength profile induces fluorescence from the scene 495 as the fluorophores within the scene 495 transition from an excited energy state caused by the illumination to a ground energy state. Additionally, the excitation light 205 may reflect from the scene 495 in the form of stray light 430, which is filtered out by filters 240A and 240B.

As illustrated in the depicted embodiment, the second image sensor 280B receives fluorescence light 220 to capture a fluorescence image representative of the scene 495 in response to the second illumination, and outputs fluorescence image data 420 in response. In some embodiments, it is appreciated that the fluorescence image may be referred to as an IR image as the wavelength profile of the fluorescence light 220 may be in the IR or near-IR (NIR) spectrum. However, the fluorescence light 220 emitted from the scene 495 may include stray light 435. The stray light 435 and the first image light 215 may have a similar or greater intensity than fluorescence light 220 and result in degradation of the fluorescence image (e.g., in the form of halos, glare, or other optical aberrations). Accordingly, second filter 240B is placed between the second image sensor 280B and the scene 495 to filter out the majority of the stray light 435, the first illumination light 205, the excitation light 210, and the first image light 215 from reaching the second image sensor 280B since fluorescence intensity is typically about 100 times less than the excitation light 205 or the first illumination light 205.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. An imaging system, comprising:
an endoscope tube;
an illumination system coupled to the endoscope tube and configured to emit first illumination light having a first wavelength profile and excitation light having an excitation wavelength profile outside of the first wavelength profile;
a first image sensor aligned with a first filter configured to pass first image light, received in response to the first illumination light, to the first image sensor and to block the excitation light;
a second image sensor aligned with a second filter configured to pass fluorescence light, emitted in response to the excitation light, to the second image sensor; and
a controller coupled to the first and second image sensors and to the illumination system, the controller including logic that, when executed, causes the imaging system to perform operations including:
simultaneously illuminating a scene with the first illumination light and the excitation light emitted from the endoscope tube; and
capturing first image data with the first image sensor and fluorescence image data with the second image sensor in response to the simultaneous illuminating.

2. The imaging system of claim 1, wherein the controller includes further logic that, when executed, causes the imaging system to perform further operations including:
adjusting an image acquisition characteristic of a selected one of the first or second image sensors independently between the first and second image sensors.

3. The imaging system of claim 2, wherein the image acquisition characteristic comprises at least one of a frame rate, an exposure time, gain, or a duty cycle.

4. The imaging system of claim 3, wherein the first illumination light comprises visible illumination light and the first image light comprises visible image light.

5. The imaging system of claim 4, wherein the controller includes further logic that, when executed, causes the imaging system to perform further operations including:
adjusting at least one of a first frame rate, a first exposure time, or a first gain of the first image sensor while holding a second frame rate, a second exposure time, and a second gain of the second image sensor constant during acquisition of a series of visible images and fluorescence images with the first and second image sensors, respectively.

6. The imaging system of claim 5 wherein the controller includes further logic that, when executed, causes the imaging system to perform further operations including:
adjusting at least one of the second frame rate, the second exposure time, or the second gain of the second image sensor while holding the first frame rate, the first exposure time, and the first gain of the first image sensor constant while acquiring a series of visible images and fluorescence images, with the first and second image sensors, respectively.

7. The imaging apparatus of claim 4, wherein the controller includes further logic that, when executed, causes the imaging system to perform further operations, including adjusting a relative intensity between the visible illumination light and the excitation light while contemporaneously emitting both.

8. The imaging apparatus of claim 4, wherein the second filter is configured to pass the fluorescence light while substantially blocking both the visible image light and the excitation light.

9. The imaging apparatus of claim 4, wherein the second filter comprises a near-infrared long-pass filter having a cutoff wavelength longer than the excitation wavelength profile.

10. The imaging apparatus of claim 1, wherein the first and second image sensors are disposed at a distal end of the endoscope tube.

11. The imaging apparatus of claim 10, wherein the first and second image sensors are oriented back-to-back at the distal end of the endoscope tube, the imaging apparatus further comprising:
first and second reflectors disposed at the distal end of the endoscope tube, wherein the first reflector is configured to direct the first image light onto the first image sensor and the second reflector is configured to direct the fluorescence light onto the second image sensor.

12. The imaging apparatus of claim 1, wherein the first and second image sensors are disposed at a proximal end of the endoscope tube, the imaging apparatus further comprising:
a beam splitter configured to receive the first image light and the fluorescence light from the endoscope tube and to direct the first image light to the first image sensor and the fluorescence light to the second image sensor.

13. A method of operation of an endoscope, the method comprising:
simultaneously illuminating a scene with visible illumination light and excitation light emitted from an endoscope tube, wherein the excitation light is outside of a visible wavelength profile of the visible illumination light;
filtering scene light received from the scene in response to the simultaneous illuminating with a first filter configured to pass visible image light and a second filter configured to pass fluorescence light; and
contemporaneously capturing visible image data in response to the visible image light incident upon a first image sensor and fluorescence image data in response to the fluorescence light incident upon a second image sensor.

14. The method of claim 13, wherein the method further comprising:
adjusting an image acquisition characteristic of a selected one of the first or second image sensors independently between the first and second image sensors.

15. The method of claim 14, wherein the image acquisition characteristic comprises at least one of a frame rate, an exposure time, a duty cycle, or a gain.

16. The method of claim 15, wherein the method further comprising:
adjusting at least a first frame rate, a first exposure time, or a first gain of the first image sensor while holding a second frame rate, a second exposure time, and a second gain of the second image sensor constant while acquiring a first series of visible images and fluorescence images with the first and second image sensors, respectively.

17. The method of claim 16, further comprising:
adjusting at least the second frame rate, the second exposure time, or the second gain of the second image sensor while holding the first frame rate, the first exposure time, and the first gain of the first image sensor constant while acquiring a second series of visible images and fluorescence images with the first and second image sensors, respectively.

18. The method of claim 16, further comprising:
adjusting a relative intensity between the visible illumination light and the excitation light while contemporaneously emitting both.

19. The method of claim 16, wherein filtering the scene light comprises passing fluorescence light with the second filter while blocking the both the first illumination light and the excitation light.

20. The method of claim 16, wherein the second filter comprises a near-infrared long-pass filter having a cutoff wavelength longer than an excitation wavelength profile.
